(19) United States
(12) Patent Application Publication
(10) Pub. No.: US 2018/0206899 A1
(43) Pub. Date: Jul. 26, 2018

(54) CUTTING TOOL FOR SURGICAL WIRES AND CABLES

(71) Applicants: This AEBI, Grenchen (CH); Simon M. BOSSHARD, Bern (CH)

(72) Inventors: This AEBI, Grenchen (CH); Simon M. BOSSHARD, Bern (CH)

(21) Appl. No.: 15/416,701

(22) Filed: Jan. 26, 2017

(51) Int. Cl. A61B 17/88; A61B 17/82

(52) U.S. Cl. CPC A61B 17/8863; A61B 17/82

(57) ABSTRACT

A device for cutting a surgical cable including a handle side including first and second gripping members pivotally attached to one another, and an intervention side including an outer tubular member, an inner tubular member within the outer tubular member having a coupling member attached on a proximal end, and a movable pinion attached on a proximal end of the coupling member. The distal end of the second gripping member is attached to a proximal end of the movable pinion and the distal ends of the outer tubular member and inner tubular member have at least two openings that are off-center but substantially aligned to allow a surgical cable or wire to pass through and to exert a shearing force on the cable or wire when the inner tubular member rotates with respect to the outer tubular member in response to compressing the first and second gripping members.

### TECHNICAL FIELD

Various exemplary embodiments disclosed herein relate generally to a cutting tool for cables.

### BACKGROUND

It is a common requirement in orthopedic surgical procedures to anchor two or more elements together, such as pieces of a bone, two or more bones, or a combination of soft tissue and bone. This has been accomplished by a number of devices, such as bone bolts that penetrate two pieces of bone and use a nut to draw the segments together, bone screws and interconnecting plates, wires circling at least two pieces of bone, or sutures into the tissue.

Often such devices require a relatively large access opening through surrounding and/or covering tissue to implant the anchoring devices. The enlarged access site may increase patient pain and lengthen recovery time. Further, in some locations it is difficult and impractical to make large access points to reach the appropriate site because of surrounding joints and vessels. Even with devices that penetrate the tissue in a substantially linear manner, i.e., lag bolts, the fracture must often be reduced before drilling and insertion of the bolt. Further, some of these devices may be difficult to use since it may be hard to reduce a fracture between two bone segments and maintain that reduction while the device is inserted. This is particularly true with small bone fragments where the use of threaded implants may tend to rotate one bone segment with respect to another, thereby creating a misalignment between the fragments.

Cerclage systems provide an alternative to implants that must penetrate the bone to achieve fixation. These systems rely on passing a cable around two segments of bone, tensioning the cable to squeeze the bone segments together, and using a cutting tool to remove the excess ends of the cable. Such cutting tools may also be used to cut sutures, wires or k-wires.

There remains a need for a minimally invasive, convenient and effective system for cutting cables used in such penetrating or cerclage systems.

### SUMMARY

A brief summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a device for cutting a surgical cable that includes: an intervention unit including first and second coaxial members each having at least two openings in a distal end; and a handle unit including an actuatable handle adapted to retain and engage the first and second coaxial members at a proximal end and to rotate the second coaxial member relative to the first. In various embodiments, the at least two openings in the distal ends of the coaxial members function as coordinating shearing structures when the handle is actuated.

Various embodiments relate to a surgical device for translating grip force to cutting force. The device includes an intervention unit including an inner tubular member disposed coaxially within an outer tubular member, and a handle unit including a housing, a coupling disposed at the distal end of the housing, a movable pinion coupled to the coupling, and a squeezable grip coupled to the movable pinion and adapted to actuate axial rotation of the movable pinion against the coupling. In one embodiment, the squeezable grip comprises a first set of teethed portions configured to engage a second set of teethed portions attached to the movable pinion.

Various embodiments relate to a device for cutting a surgical cable including a handle unit including first and second gripping members pivotally attached to one another, first and second handle grips attached at proximal ends of the first and second gripping members, respectively, and an intervention unit including an outer tubular member attached on a proximal end of the outer tubular member to a distal end of the first gripping member, an inner tubular member located within the outer tubular member and having a coupling member radially attached on a circumference of a proximal end, and a movable pinion radially attached on a circumference of a proximal end of the coupling member. In various embodiments, a distal end of the second gripping member is attached to a proximal end of the movable pinion and the distal ends of the outer tubular member and inner tubular member have at least two openings that are off-center but substantially aligned such as to allow a surgical cable or wire to pass through and to exert a shearing force on the cable or wire, when the inner tubular member rotates with respect to the outer tubular member in response to compressing the first and second handle grips.

Various embodiments relate to a device for cutting a surgical cable that includes: a cutting unit including outer and inner tubular members each having at least two openings in a distal end; and a handle unit including a trigger-handle adapted to retain and engage the outer and inner tubular members at a proximal end and to rotate the inner tubular member relative to the outer tubular member. In various embodiments, the at least two openings in the distal ends of the tubular members function as coordinating shearing structures when the trigger-handle is actuated.

Various embodiments relate to a device for cutting a surgical cable including a handle unit including a housing and a trigger-handle pivotally attached to one another, first and second handle grips formed on proximal ends of the housing and trigger-handle, respectively, and a cutting unit including an outer tubular member attached on a proximal end to a distal end of the handle unit, an inner tubular member within the outer tubular member and having a coupling member radially attached on a circumference of a proximal end, and a movable pinion radially attached on a circumference of a proximal end of the coupling member. In various embodiments, a part of the trigger-handle interacts with a part of the proximal end of the movable pinion and the distal ends of the outer tubular member and inner tubular member have at least two openings that are off-center but substantially aligned such as to allow a surgical cable or wire to pass through and to exert a shearing force on the cable or wire, when the inner tubular member rotates with respect to the outer tubular member in response to pulling the trigger-handle to the housing.

Various embodiments relate to a surgical device for translating gripping force to cutting force. The device includes a cutting unit including an inner tubular member disposed coaxially within an outer tubular member, a handle unit, including a movable pinion adjacent to the inner and outer tubular members along a portion of a length of the inner tubular member and proximate a first end of the tubular members, the handle unit further including a housing and a trigger-handle pivotally connected to the housing and adapted to actuate the movable pinion when the trigger-handle is actuated. In one embodiment, the movable pinion has a plurality of teethed portions configured to interact with a plurality of teethed portions attached to the trigger-handle.

Various embodiments relate to a pistol-shaped device for cutting a surgical cable that may be one-hand operable and may be able to cut cables up to a specific diameter and strength.

Various embodiments relate to a device for cutting a surgical cable that may include the following parts: a housing, a trigger-handle pivotally connected to the housing over a bearing bolt, a coupling part connected to the distal end of the housing, an inner tubular member and an outer tubular member mounted to the coupling part with a connection nut, a pinion that transfers torque between the trigger-handle and the inner cutting tube, a guiding tube mounted between the proximal end of the pinion and the proximal end of the housing and a leaf spring that resets the position of the trigger-handle relatively to the housing.

In various embodiments the distal end of the device includes at least two equivalent cutting holes where a surgical cable may be inserted when the device is in its reset position. In various embodiments, a cable may be guided through one of the at least two cutting holes wherein the device may be positioned relatively to the length of the cable at the required cutting location. After cutting, the loose cable and the device may be removed. In various embodiments the cable may be pushed all the way through the device until the cable leaves the device at its back end.

In various embodiments, the trigger-handle may be pulled back to a mechanical end stop. In one embodiment, once the cable is severed, the loose proximal end of the cable may be removable through the back end of the device.

Various embodiments relate to a device for cutting a surgical cable wherein the tubular members may be removed from the coupling part for washing and sterilization.

It is contemplated that various combinations of the embodiments described herein may be made resulting in additional embodiments that are within the scope of the invention described herein.

### BRIEF DESCRIPTION OF THE DRAWINGS

In order to better understand various exemplary embodiments, reference is made to the accompanying drawings, wherein:

FIGS. 1A-D illustrate a perspective view, side view, distal view and top view of one embodiment of a device for cutting surgical wire or cable.

FIG. 2 illustrates an exploded view of the device of FIG. 1.

FIG. 3A illustrates a cross-sectional side view of the device of FIG. 1.

FIG. 3B illustrates a partial cross-sectional top view of the device of FIG. 1.

FIGS. 4A-C illustrate a perspective view, distal view and proximal view of the outer tubular member described in FIG. 2.

FIGS. 4D-F illustrate a perspective view, distal view and proximal view of the inner tubular member described in FIG. 2.

FIGS. 5A-B illustrate a cross sectional side view and a distal view of the coupling part described in FIG. 2.

FIGS. 6A-B illustrate a side view and a distal view of the pinion described in FIG. 2.

FIG. 7 illustrates a side view of the trigger-handle described in FIG. 2.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art, and are to be construed as being without limitation to such specifically recited examples and conditions. Additionally, the term, “or,” as used herein, refers to a non-exclusive or (i e, and/or), unless otherwise indicated (e.g., “or else” or “or in the alternative”). Also, the various embodiments described herein are not necessarily mutually exclusive, as some embodiments can be combined with one or more other embodiments to form new embodiments.

Embodiments of a cutting tool 100 for cutting a surgical cable are described below. The cutting tool 100 may be used for cutting different cables up to 2.0 mm in diameter, independent of cable material and strand type. The cutting tool 100 additionally enables in-line cutting of the cable without any change in length of the cutting tool 100 during cutting and also enables minimally invasive access to the appropriate cutting position. The cutting tool 100 further enables faster and easier handling and one-hand use. In one embodiment, the cable guides the cutting tool 100 to the appropriate cutting position.

The cutting tool 100 may include two main assembly units: (1) a cutting unit 300 and (2) a handle unit 200. In some embodiments, the cutting unit 300 allows for in-line cutting of the cable without any bending or pulling on the cable. The cutting unit 300 may also allow for installation of other instruments or handle units. The cutting unit 300 may allow for an immediate shearing movement through axial rotation of an inner tubular member 320 against an outer tubular member 310 through the following sequence: reset position>cable cutting>end position.

The cutting unit 300 may include an inner tubular member 320, an outer tubular member 310, and a connection nut 330. The inner tubular member 320 may be made from one single piece with at least two cutting holes in the distal front end, symmetrically arranged around an axis. The outer tubular member 310 may also be made from one single piece with at least two cutting holes in the distal front end, aligned with the holes of the inner tubular member 320 in the reset position.

The cutting unit 300 in the reset position state allows for insertion of the cable all the way through the cutting tool 100. In the cable cutting state, the cable is blocked from any axial movement in the distal or proximal direction relative to the cutting tool 100. The cutting unit 300 in the end position allows for removal of the cut piece of cable out of the cutting tool 100 in a proximal direction and removal of the cutting tool 100 from the distal cut piece.

The cutting unit 300 may work and move radially in a clockwise and counterclockwise direction. The cutting unit 300 and handle unit 200 may be quickly coupled over a threaded joint. The cutting unit 300, which is at high risk of contamination during surgery, may be completely decomposable for reprocessing. The handle unit 200 with all other parts has a lower risk of contamination during surgery. The handle unit 200 may be non-decomposable and may be reprocessed as one single element.

The mechanism of cutting employed by the cutting tool 100 may be realized through an axial rotation of an inner tubular member 320 against an outer tubular member 310. The cutting forces result in shearing stresses and axial stresses; whereas the axial stresses are neutralized between the proximal end faces of the inner tubular member 320, the outer tubular member 310, the connection nut 330, and the coupling socket 262 of the coupling part 260. In some embodiments, the clearance between the outside cutting face 323 of the inner tubular member 320 and the inside cutting face 313 of the outer tubular member 310 may be smaller than the diameter of a single braid of the cable.

The setup of the cutting mechanism may be realized with at least two equivalent cutting holes which prolong the lifetime expectancy of the cutting device by providing a coincident choice of the cutting hole and reassembly after reprocessing. Furthermore, the choice of one of at least two possible cutting holes increases the chances of obtaining a good position for the instrument relative to the cutting site.

In some embodiments, the handle unit 200 of the cutting tool 100 may be manufactured by investment casting. The cutting unit 300 and the handle unit 200 may be configured to allow one-handed use by both left and right handed users.

The handle unit 200 allows for coincident radial alignment of the inner tubular member 320 relative to the outer tubular member 310 in multiple positions all leading to alignment of the cutting holes. The handle unit 200 also allows for installation of other instrument or intervention units at its distal end. The handle unit 200 further allows for an immediate rotation movement, i.e., axial rotation, of a pinion 250 against a coupling part 260.

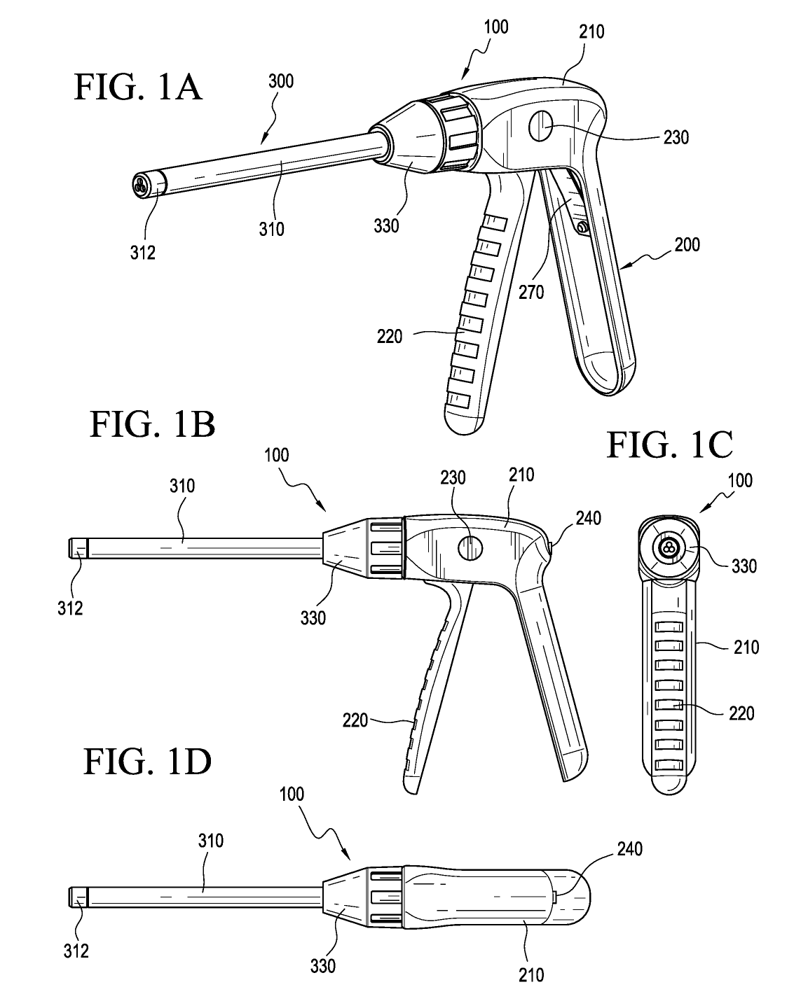

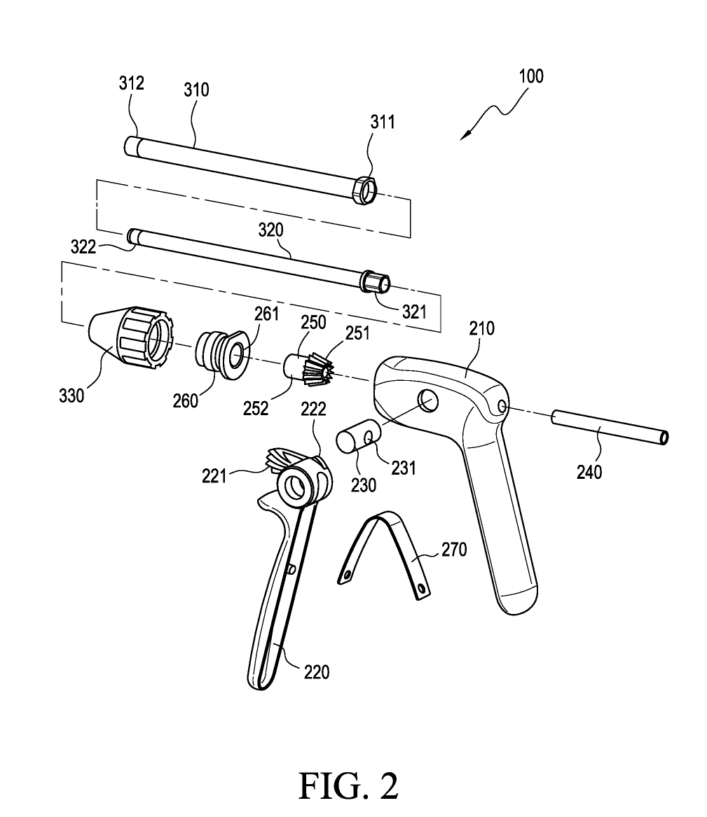

FIGS. 1A-D illustrate four different views of one embodiment of a cutting tool 100 for cutting surgical wire or cable. The cutting tool 100 includes a handle unit 200 and a cutting unit 300. The handle unit 200 includes a trigger-handle 220 and a housing 210 which are pivotally connected by a bearing bolt 230. The cutting unit 300 includes an inner tubular member 320 (see FIG. 2), an outer tubular member 310 and a connection nut 330 that assists in securing the inner tubular member 320 and the outer tubular member 310 to the handle unit 200.

The housing 210 may be made from surgical grade stainless steel, plastic, polymers, or other suitable materials or combinations thereof. The housing 210 may be formed by casting, machining, polishing, and/or other methods or some combinations thereof. The exact dimensions of the housing 210 may vary with the application, but the size will generally be such that an operator may maintain a comfortable and effective grip on the cutting tool 100 during operation.

The trigger-handle 220 may be made from similar or different materials than the housing 210, including surgical grade stainless steel, plastic, polymers, or other suitable materials or combinations thereof. The trigger-handle 220 may be formed by casting, machining, polishing and/or other methods or combinations thereof. The dimensions of the trigger-handle 220 may be chosen to match those of the housing 210, and may be chosen such as to provide an effective and comfortable grip for the operator. As will be described in greater detail below, the trigger-handle 220 may be equipped with a first teethed portion 221 adapted to engage a moveable pinion 250 that contains a second teethed portion 251 (see FIG. 2).

The housing 210 and trigger-handle 220 are pivotally connected by a bearing bolt 230. The bearing bolt 230 allows the housing 210 and the trigger-handle 220 to move in the same plane of motion in a scissor-like fashion. The bearing bolt 230 may comprise the same or different materials than the housing 210 and the trigger-handle 220. For example, the bearing bolt 230 may be surgical grade stainless steel or another suitable material.

The outer tubular member 310 may be formed separately from the housing 210 and then attached together. The outer tubular member 310 may be formed of similar or different materials than the other components of the cutting tool 100. The outer tubular member 310 may be formed by casting, machining, and/or other methods. As will be described in greater detail below, the outer tubular member 310 may be substantially hollow to accommodate an inner tubular member 320 (see FIG. 2).

A connection nut 330 may assist in securing the outer tubular member 310 and the inner tubular member 320 to the handle unit 200. The connection nut 330 may be composed of different or similar materials than the other components of the cutting tool 100. The connection nut 330 may comprise surgical grade stainless steel, another metal, plastic, polymers, other suitable materials, and/or combinations thereof. The connection nut 330 may be formed by machining or casting, for example.

FIG. 2 illustrates an exploded view of one embodiment of a cutting tool 100 for cutting a surgical cable. Here it may be seen that, in the handle unit 200, the trigger-handle 220 attaches to the housing 210 pivotally by the bearing bolt 230. The bearing bolt 230 may be held in place by a guiding tube 240 that is inserted through a cross hole 231 in the bearing bolt 230. The bearing bolt 230 may alternatively be connected to the housing 210 through a press fit or weld seam. The guiding tube 240 may be used to guide the cable from the proximal end of the pinion 250, through the trigger-handle 220 and the bearing bolt 230 to the proximal end of the cutting tool 100.

The trigger-handle 220 may also be seen here to have a first teethed portion 221. The handle unit 200 further includes a leaf spring 270 that may attach to the housing 210 and the trigger-handle 220. The leaf spring 270 causes the trigger-handle 220 to revert to a reset position when a user releases the trigger-handle 220.

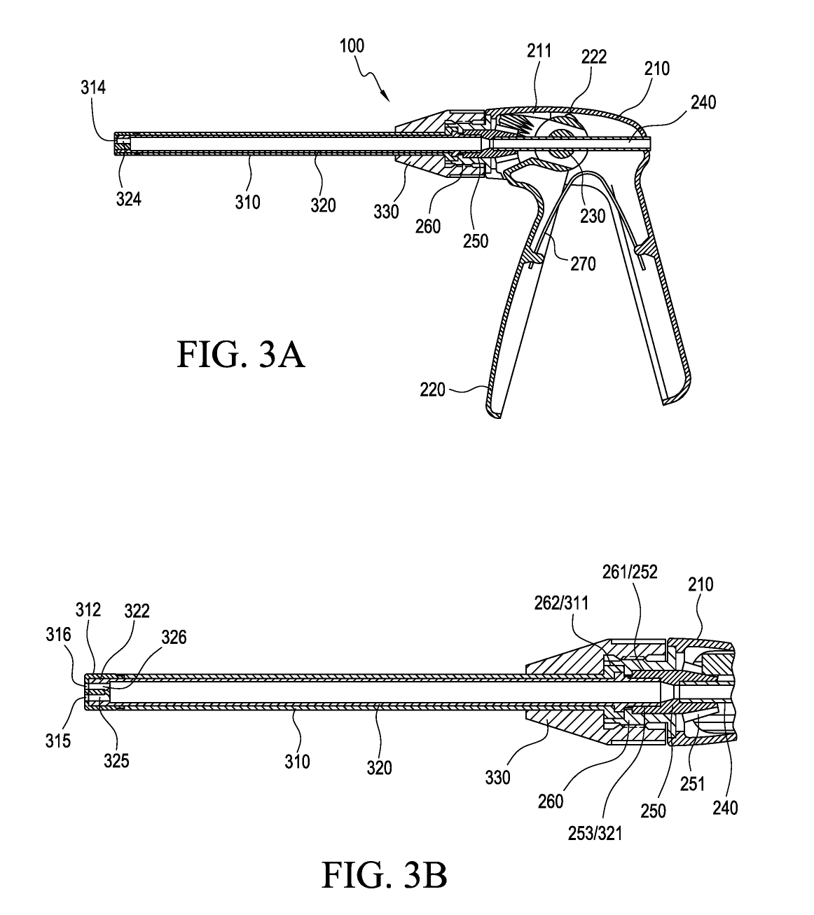

In regard to the cutting unit 300, the first teethed portion 221 of the trigger-handle 220 may be configured to engage a second teethed portion 251 positioned on the proximal end of a movable pinion 250. The trigger-handle 220, when pulled inward toward the housing 210, is configured to rotate the movable pinion 250. The stop tooth 222 engages an end stop 211 (see FIG. 3A) inside the housing 210. The movable pinion 250 may be made from similar or different materials than the components previously described. In one embodiment, the pinion 250 comprises surgical grade stainless steel. The pinion 250 may be formed by casting, machining, and/or other methods.

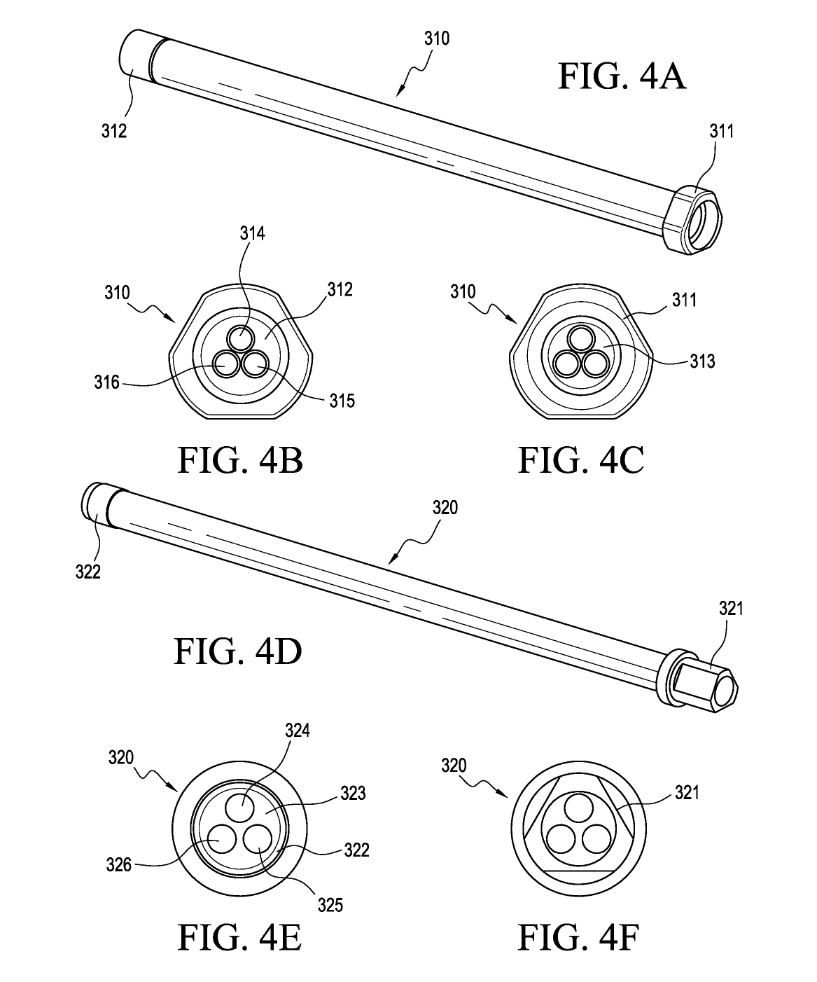

An inner tubular member 320 may be coaxially fitted into the substantially hollow outer tubular member 310. The inner tubular member 320 may contain a cutting end 322 with an outside cutting face 323 including at least two cutting holes in its distal end (see FIGS. 4A-F). The central opening of the inner tubular member 320 allows a surgical cable to pass through at least one of the cutting holes. The inner tubular member 320 may be made from the same or different materials than the components previously described. The inner tubular member 320 may be made from surgical grade stainless steel, for example. The inner tubular member 320 may be formed by casting, machining, or other methods.

The inner tubular member 320 and the movable pinion 250 may be secured together using a coupling part 260. The coupling part 260 may be made from similar or different materials than the components previously described. The coupling part 260 may be made from surgical grade stainless steel, for example. The coupling part 260 may be formed by casting, machining, or other methods. The coupling part 260 may be fixed with a weld seam to the housing 210. The housing 210 may be manufactured together with the coupling part 260 as one element.

A connection nut 330 is configured to engage the coupling part 260 and secure the inner tubular member 320, and the outer tubular member 310 to the coupling part 260 and the housing 210.

FIG. 3A illustrates a cross-sectional side view of one embodiment of cutting tool 100 for cutting surgical wire or cable. In FIG. 3A, the cutting tool 100 is shown fully assembled. A first inner cutting hole 324 is present at the distal end of the inner tubular member 320 and is aligned with a first outer cutting hole 314 of the distal end of the outer tubular member 310. The cutting holes 314, 324 allow for the passing of a surgical cable or wire when cutting tool 100 is in the reset position.

FIG. 3B illustrates a cross-sectional top view of the distal part of the cutting tool 100 wherein a second inner cutting hole 325 and a third inner cutting hole 326 are present on the distal end of the inner tubular member 320 and aligned with a second outer cutting hole 315 and a third outer cutting hole 316 of the outer tubular member 310. It is possible to change the alignment of all outer cutting holes 314, 315 and 316 with respect to all inner cutting holes 324, 325 and 326 with no effect on properties or handling of the cutting tool 100. This change of alignment will occur coincidentally during disassembly/assembly actions and thus, prolongs the lifetime of the cutting unit 300 by the number of implemented cutting holes.

The cutting holes 314, 315, 316, 324, 325, 326 are shown as circular, but the cutting unit 300 is not so limited and other shapes for the cutting holes may be utilized. The cable or wire (not shown) may pass through the hollow portion of the inner tubular member 320 and out the back of the cutting tool 100. The dimensions of the inner tubular member 320 and the outer tubular member 310 may be chosen such that when assembled, the outside cutting face 323 of the inner tubular member 320 is substantially flush against the inside cutting face 313 of the outer tubular member 310. FIGS. 3A and 3B illustrate the cutting tool 100 in its reset position, which is an “open” state that allows a wire or surgical cable to pass.

FIG. 4A illustrates a perspective view of an embodiment of the outer tubular member 310 including the coupling end 311 and the cutting end 312. FIG. 4B illustrates a distal view of an embodiment of the cutting end 312 of the outer tubular member 310, which is characterized by the three outer cutting holes 314, 315, 316. FIG. 4C illustrates a proximal view of an embodiment of the coupling end 311 of the outer tubular member 310, which is characterized by a triangular shape. Other embodiments of the outer tubular member 310 may be characterized by a coupling end 311 having a different shape. The shape of the coupling end 311 of the outer tubular member 310 allows for rotational force to be transmitted from the coupling end 311 of the outer tubular member 310 to the coupling socket 262 of the coupling part 260 (shown in FIG. 3B) during cutting of a cable. Other suitable shapes of the coupling end 311 may include a square, a circle with one or more flat portions, a circular shape with a key, or the like.

FIG. 4D illustrates a perspective view of an embodiment of the inner tubular member 320 including the coupling end 321 and the cutting end 322. FIG. 4E illustrates a distal view of an embodiment of the cutting end 322 of the inner tubular member 320, which is characterized by the outside cutting face 323 and the three inner cutting holes 324, 325, 326. FIG. 4F illustrates a proximal view of an embodiment of the coupling end 321 of the inner tubular member 320, which is characterized by a triangular shape. Other embodiments of the inner tubular member 320 may be characterized by a coupling end 321 having a different shape. The shape of the coupling end 321 of the inner tubular member 320 allows for rotational force to be transmitted from the coupling socket 253 of the pinion 250 to the coupling end 321 of the inner tubular member 320 (shown in FIG. 3B) during cutting of a cable. Other suitable shapes of the coupling end 321 may include a square, a circle with one or more flat portions, a circular shape with a key, or the like.

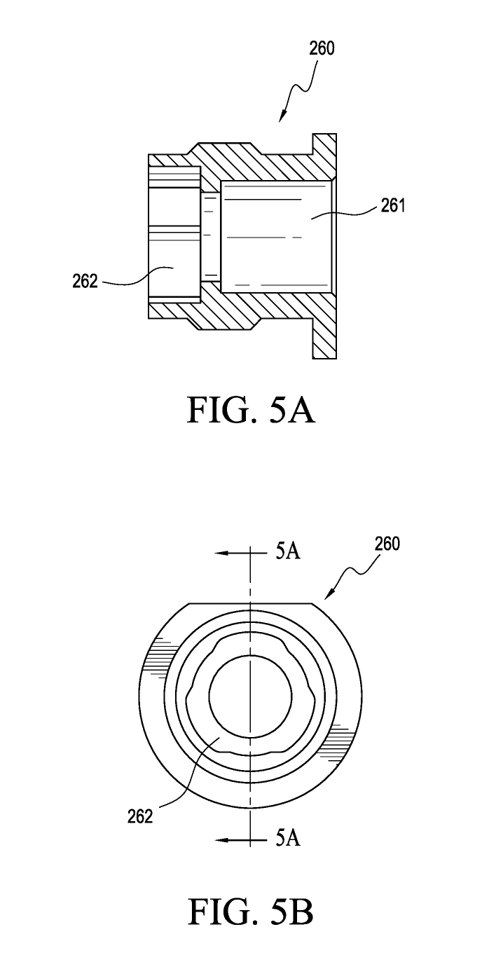

FIG. 5A illustrates a cross-sectional side view of an embodiment of the coupling part 260 of the cutting tool 100. The coupling part 260 includes a coupling socket 262 to receive and rotationally fix the coupling end 311 of the outer tubular member 310 and a bearing socket 261 to receive the bearing end 252 of the pinion 250. FIG. 5B illustrates a distal view of the coupling part 260 wherein the coupling socket 262 is characterized by a triangular shape. Other embodiments of the coupling part 260 may be characterized by a coupling socket 262 having a different shape. Other suitable shapes may include a square, a circle with one or more flat portions, a circular shape with a key, or the like.

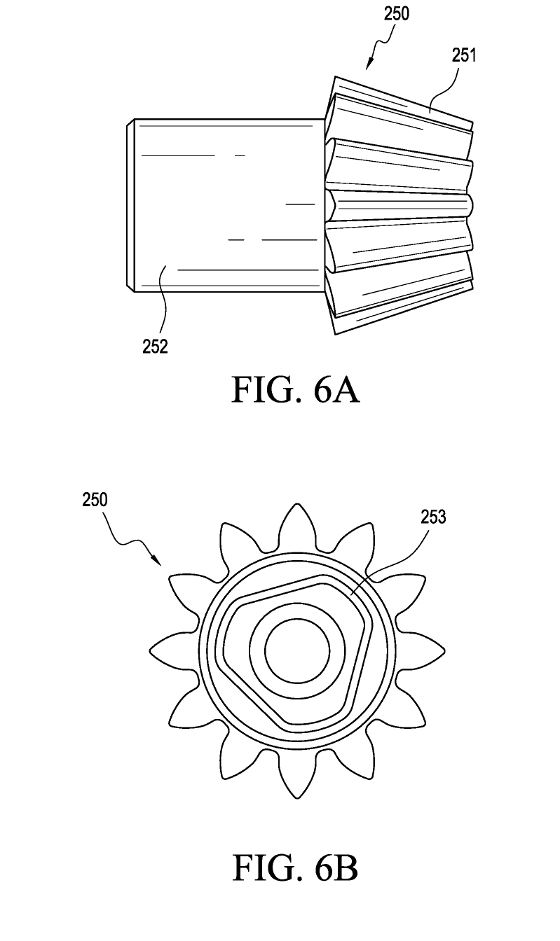

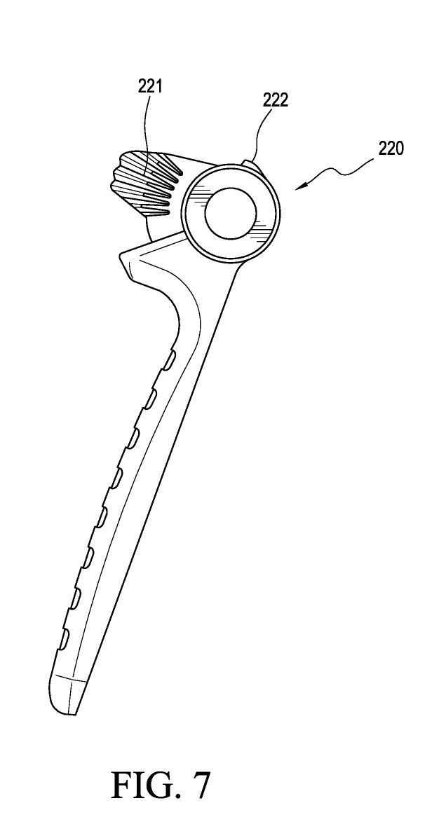

FIG. 6A illustrates a side view of an embodiment of the movable pinion 250 of the cutting tool 100. The movable pinion 250 includes a second teethed portion 251 and a bearing end 252. FIG. 6B illustrates a distal view of an embodiment of the movable pinion 250, including the coupling socket 253 which, like the outer tubular member 310 and inner tubular member 320, may be characterized by a triangular shape. Other embodiments of the pinion 250 may be characterized by a coupling socket 253 having a different shape. The coupling socket 253 of the movable pinion 250 is configured to mate with the coupling end 321 of the inner tubular member 320. The second teethed portion 251 of the movable pinion 250, is configured to engage a first teethed portion 221 of the trigger-handle 220, which is illustrated in more detail in FIG. 7.

The cutting tool 100 described in FIGS. 1-7 may be operated by pulling the trigger-handle 220 inward in the direction of the housing 210 wherein the first teethed portion 221 of the trigger-handle 220 engages with the second teethed portion 251 of the pinion 250 and rotates the pinion 250 until the stop tooth 222 at the trigger-handle 220 is in contact with the end stop 211 (see FIG. 3A) in the housing 210. The pinion 250, which is engaged with the coupling end 321 of the inner tubular member 320, then rotates the inner tubular member 320 coaxially with respect to the outer tubular member 310. The outer tubular member 310 may be held stationary as a result of its engagement of the coupling end 311 in the coupling socket 262 of the coupling part 260.

It should be appreciated by those skilled in the art that any diagrams or schematic drawings herein represent conceptual views of illustrative structures embodying the principles of the invention.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be effected while remaining within the spirit and scope of the invention. Further, various elements from the various embodiments may be combined to form other embodiments that are within the spirit and scope of the invention. Accordingly, the foregoing disclosure, description, and figures are for illustrative purposes only and do not in any way limit the invention, which is defined only by the claims.

1. A device for cutting a surgical cable, comprising:
an intervention unit comprising first and second coaxial members each comprising at least two openings in a distal end; and
a handle unit comprising an actuatable trigger handle adapted to retain and engage the first and second coaxial members at a proximal end and to rotate the second coaxial member relative to the first;
wherein the at least two openings in the distal ends of the coaxial members function as coordinating shearing structures when the actuatable trigger handle is actuated.

2. The device of claim 1, wherein the first and second coaxial members are configured with the second coaxial member inside the first, and the distal end of the second coaxial member abutting the first coaxial member along the longitudinal axis.

3. The device of claim 1, wherein the first and second coaxial members each comprise three openings in the distal end.

4. The device of claim 3, wherein the alignment of the three openings in one coaxial member is changeable with respect to the three openings in the other coaxial member.

5. The device of claim 3, wherein the proximal ends of the first coaxial member and the second coaxial member are characterized by a first shape to mate with a congruent shape on a pinion or coupling that allows for a rotational force to be transmitted by the mated shapes.

6. The device of claim 1, wherein the device is actuated by rotation of the second coaxial member relative to the first when the actuatable trigger handle is actuated.

7. The device of claim 6, wherein the second coaxial member is attached to a pinion and wherein the actuatable trigger handle comprises a first set of teeth that is configured to engage a second set of teeth attached to the pinion.

8. The device of claim 1, wherein the intervention unit is removably attached to the handle unit.

9. The device of claim 8, wherein the intervention unit is removably attached to the handle unit using a connection nut.

10. The device of claim 1, wherein the handle unit allows for coincident radial alignment of the first coaxial member relative to the second coaxial member in multiple positions all leading to alignment of the at least two openings in the distal ends of the coaxial members.

11. The device of claim 8, wherein the at least two openings are arranged in such a way that a change of alignment occurs coincidentally during each re-attachment of the intervention unit to the handle unit.

12. The device of claim 1, wherein the length of the device remains constant when the actuatable trigger handle is actuated.

13. A surgical device for translating grip force to cutting force comprising:
an intervention unit comprising
an inner tubular member disposed coaxially within an outer tubular member; and
a handle unit comprising
a housing;
a coupling disposed at the distal end of the housing;
a movable pinion coupled to the coupling; and
a squeezable trigger handle coupled to the movable pinion and adapted to actuate axial rotation of the movable pinion against the coupling;
wherein the squeezable trigger handle comprises a first set of teethed portions configured to engage a second set of teethed portions attached to the movable pinion.

14. The device of claim 13, wherein the inner tubular member and outer tubular member each have at least two openings on a distal end, the openings configured to translate a shear force between them when the inner tubular member and outer tubular member are rotated relative to each other.

15. The device of claim 13, wherein the inner and outer tubular member each have three openings on a distal end.

16. The device of claim 15, wherein the alignment of the three openings in one tubular member is changeable with respect to the three openings in the other tubular member.

17. The device of claim 15, wherein the proximal ends of the inner and outer tubular members are characterized by a first shape to mate with a congruent shape on the pinion or coupling that allows for a rotational force to be transmitted by the mated shapes.

18. The device of claim 13, wherein the handle unit comprises a first stationary member attached to the coupling member and a second movable member configured to engage the movable pinion, the first and second members being pivotally attached, and wherein the handle unit is configured to be positioned outside the patient.

19. The device of claim 14, wherein the intervention unit is removably attached to the handle unit.

20. The device of claim 19, wherein the intervention unit is removably attached to the handle unit using a connection nut.

21. The device of claim 14, wherein the handle unit allows for coincident radial alignment of the inner tubular member relative to the outer tubular member in multiple positions all leading to alignment of the at least two openings in the distal ends of the tubular members.

22. The device of claim 19, wherein the at least two openings are arranged in such a way that a change of alignment occurs coincidentally during each re-attachment of the intervention unit to the handle unit.

23. The device of claim 13, wherein the length of the device remains constant when the trigger handle is squeezed.

24. A device for cutting a surgical cable comprising:
first and second gripping members pivotally attached to one another; wherein the second gripping member comprises a trigger handle;
first and second handle grips attached at proximal ends of the first and second gripping members, respectively;
an outer tubular member attached on a proximal end of the outer tubular member to a distal end of the first gripping member;
an inner tubular member located within the outer tubular member and having at least one coupling member attached on a circumference of a proximal end; and
a movable pinion attached on a circumference of the proximal end of the coupling member;
wherein a distal end of the second gripping member is engaged with a proximal end of the movable pinion;
wherein the distal end of the inner tubular member abuts an internal surface of the outer tubular member proximal the distal end thereof, the outer tubular member and inner tubular member comprising at least two openings at the distal ends thereof that are substantially aligned to form a passage allowing a surgical cable to pass through and to exert a shearing force on the cable when the inner tubular member rotates with respect to the outer tubular member in response to compressing the first and second handle grips.

25. The device of claim 24, wherein the outer and inner tubular members each comprise three openings in the distal end.

26. The device of claim 25, wherein the alignment of the three openings in one tubular member is changeable with respect to the three openings in the other tubular member.

27. The device of claim 25, wherein the proximal ends of the outer and inner tubular members are characterized by a first shape that mates with a congruent shape on the pinion or coupling that allows for a rotational force to be transmitted by the mated shapes.

28. The device of claim 24, wherein the second gripping member comprises a first set of teeth that is configured to engage a second set of teeth attached to the movable pinion.

29. The device of claim 24, wherein the inner and outer tubular members are removably attached to the first and second gripping members.

30. The device of claim 29, wherein the inner and outer tubular members are removably attached to the first and second gripping members using a connection nut.

31. The device of claim 24, wherein the device allows for coincident radial alignment of the inner tubular member relative to the outer tubular member in multiple positions all leading to alignment of the at least two openings in the distal ends of the tubular members.

32. The device of claim 29, wherein the at least two openings are arranged in such a way that a change of alignment occurs coincidentally during each re-attachment of the intervention unit to the handle unit.

33. The device of claim 24, wherein the length of the device remains constant when the first and second handle grips are compressed.
